(19) United States
(12) Patent Application Publication
(10) Pub. No.: US 2012/0143071 A1
(43) Pub. Date: Jun. 7, 2012

(54) ALGORITHMS FOR CALCULATION OF PHYSIOLOGIC PARAMETERS FROM NONINVASIVE PHOTOPLETHYSMOGRAPHIC SENSOR MEASUREMENTS OF AWAKE ANIMALS

(75) Inventors: Bernard F. Hete, Kittanning, PA (US); Eric J. Ayers, Aliquippa, PA (US); Eric W. Starr, Allison Park, PA (US)

(73) Assignee: STARR LIFE SCIENCES CORP., Oakmont, PA (US)

(21) Appl. No.: 13/156,489

(22) Filed: Jun. 9, 2011

Related U.S. Application Data
(63) Continuation of application No. PCT/US09/67406, filed on Dec. 9, 2009.
(60) Provisional application No. 61/121,162, filed on Dec. 9, 2008.

(51) Int. Cl. A61B 5/02

(52) U.S. Cl. 600/504

(57) ABSTRACT

A noninvasive photoplethysmographic sensor system for mobile animals such as small rodents, namely rats and mice is useful such as in a laboratory research environment. The noninvasive photoplethysmographic sensor for mobile animals such as small rodents utilizes multiple FFT's in the processing of the phtotoplethysmograophic signal, where each FFT has a different time record of the signals such as a number of points, or a zero padded FFTs. The noninvasive photoplethysmographic sensor for mobile animals provides actigraphy measurements for the animal.

### RELATED APPLICATIONS

The present invention is a continuation of international patent application serial number PCT/US2009/067406 filed Dec. 9, 2009 and which published as Publication number WO 2010/068713 which is incorporated herein by reference. International patent application serial number PCT/US2009/067406 entitled “Algorithms for Calculation of Physiologic Parameters from Noninvasive Photoplethysmographic Sensor Measurements of Awake Animals” claims priority of U.S. Provisional Patent Application Ser. No. 61/121,162 entitled “Algorithms for Calculation of Physiologic Parameters from Noninvasive Photoplethysmographic Sensor Measurements of Awake Animals” filed Dec. 9, 2008.

### BACKGROUND OF THE INVENTION

1. Field of the Invention

The present invention relates to photoplethysmographic readings for animal research and more particularly, the present invention is directed to a noninvasive photoplethysmographic sensor for mobile awake animals such as small rodents.

2. Background Information

A photoplethysmograph is an optically obtained plethysmograph, which, generically, is a measurement of changes in volume within an organ whole body, usually resulting from fluctuations in the amount of blood or air that the organ contains. A photoplethysmograph is often obtained by using a pulse oximeter. A conventional pulse oximeter monitors the perfusion of blood to the dermis and subcutaneous tissue of the skin. Pulse oximetry is a non invasive method that allows for the monitoring of the oxygenation of a subject's blood, generally a human or animal patient or an animal (or possibly human) research subject. The patient/research distinction is particularly important in animals where the data gathering is the primary focus, as opposed to care giving, and where the physiologic data being obtained may, necessarily, be at extreme boundaries for the animal.

A short history of pulse oximetry may be elucidating. It has been reported Mr. Matthes developed, in 1935, the first 2-wavelength earlobe O₂ saturation meter with red and green filters that were later switched to red and infrared filters. In 1949 an inventor Wood added a pressure capsule to squeeze blood out of the earlobe to obtain zero setting in an effort to obtain absolute O₂ saturation value when blood was readmitted. The concept is similar to today's conventional pulse oximetry but suffered due to unstable photocells and light sources and the method was not used clinically. In 1964 an inventor Shaw assembled the first absolute reading ear oximeter by using eight wavelengths of light which was commercialized by Hewlett Packard. This use was limited to pulmonary functions due to cost and size.

Effectively, modern pulse oximetry was developed in 1972, by Aoyagi at Nihon Kohden using the ratio of red to infrared light absorption of pulsating components at the measuring site, and this design was commercialized by BIOX/Ohmeda in 1981 and Nellcor, Inc. in 1983. Prior to the introduction of these commercial pulse oximeters, a patient's oxygenation was determined by a painful arterial blood gas, a single point measure which typically took a minimum of 20-30 minutes processing by a laboratory. It is worthy to note that in the absence of oxygenation, damage to the human brain starts in 5 minutes with brain death in a human beginning in another 10-15 minutes. Prior to its introduction, studies in anesthesia journals estimated US patient mortality as a consequence of undetected hypoxemia at 2,000 to 10,000 deaths per year, with no known estimate of patient morbidity. Pulse oximetry has become a standard of care for human patients since about 1987.

Pulse oximetry has been a critical research tool for obtaining associated physiologic parameters in humans and animals beginning soon after rapid pulse oximetry became practical.

In pulse oximetry a sensor is placed on a thin part of the subject's anatomy, such as a fingertip or earlobe in humans, or in the case of a neonate, across a foot. For transmittance readings two wavelengths of light, generally red and infrared wavelengths, are passed from one side to the other. Changing absorbance of each of the two wavelengths is measured, allowing determination of the absorbance due to the pulsing arterial alone, excluding venous blood, skin, bone, muscle, fat, etc. Based upon the ratio of changing absorbance of the red and infrared light caused by the difference in color between oxygen-bound (bright red) and oxygen unbound (dark red or blue, in severe cases) blood hemoglobin, a measure of oxygenation (the percent of hemoglobin molecules bound with oxygen molecules) can be made.

The measured signals of pulse oximeters are also utilized to determine other physical parameters of the subjects, such as heart rate (AKA pulse rate). Starr Life Sciences, Inc. has utilized pulse oximetry measurements to calculate other physiologic parameters such as breath rate, pulse distension, and breath distention, which can be particularly useful in various research applications.

Regarding human and animal pulse oximetry, the underlying theory of operation remains the same. However, consideration must be made for the particular subject or range of subjects in the design of the pulse oximeter, for example the sensor must fit the desired subject (e.g., a medical pulse oximeter for an adult human finger simply will not adequately fit onto a mouse finger or paw; and regarding signal processing the signal areas that are merely noise in a human application can represent signals of interest in animal applications due to the subject physiology). Consequently there can be significant design considerations in developing a pulse oximeter for small mammals or for neonates or for adult humans, but, again the underlying theory of operation remains substantially the same.

In addressing animal pulse oximetry, particularly for small rodents, one approach has been to modify existing human or neonate oximeters for use with rodents. This approach has proven impractical as the human based systems can only stretch so far and this approach has limited the use of such adapted oximeters. For example, these adapted human oximeters for animals have an upper limit of heart range of around 400 or 450 beats per minute which is insufficient to address mice that have a conventional heart rate of 400-800 beats per minute. Starr Life Sciences has designed a small mammal oximeter from the ground up, rather than an adapted human model, that has effective heart rate measurements up to 900 beats per minute, and this is commercially available under the Mouse Ox™ oximeter brand since 2005.

In the field of pulse oximetry in humans, U.S. Pat. No. 5,005,573 discloses an oximetry device in an endotracheal tube to enable “more accurate” and “more quickly responsive” oximetry measurements to be made through the patient's neck an to enable continual monitoring of the tube position within the trachea. Although this placement can provide improved oximetry measurements, it is much more invasive than conventional external pulse oximeters that have been placed on human fingers, toes and earlobes. Further, endotracheal tube placement is impractical or mobile animal studies and for studies of small animals such as rodents (e.g. mice and rats).

Regarding the present disclosure the International Search Report in the parent application identified the Assignee's U.S. Published application 2008-0167564, U.S. Published application 2005-0065414, U.S. Published application 2004-0039273, and U.S. Published application 2006-0211930 as “documents defining the general state of the art” and are cited here for convenience. U.S. Pat. No. 7,139,599 is identified as related to and possible equivalent of U.S. Published application 2004-0039273 and U.S. Pat. No. 7,020,507 is identified as related to and possible equivalent of U.S. Published application 2006-0211930.

It is an object of the present invention to address the deficiencies of the prior art discussed above and to do so in an efficient, cost effective manner.

### SUMMARY OF THE INVENTION

The various embodiments and examples of the present invention as presented herein are understood to be illustrative of the present invention and not restrictive thereof and are non-limiting with respect to the scope of the invention.

According to one non-limiting embodiment of the present invention, a noninvasive photoplethysmographic sensor platform for mobile animals such as small rodents, namely rats and mice, is provided on an adjustable animal neck collar or neck clip. According to one non-limiting embodiment of the present invention, the present invention utilizes multiple FFT's in the processing of the phtotoplethysmograophic signal, where each FFT has a different amount of signal history, such as having different number of points. This signal processing procedure increases the ability to differentiate nuances in the base phtotoplethysmograophic signal. In the application described here, the goal of using multiple FFTs is to provide more information that can be used to deduce heart rate of a very noisy time-domain signal.

A further non-limiting embodiment of the invention provides a noninvasive photoplethysmographic sensor platform for mobile animals comprising using motion detection to provide actigraphy (motion) measurements of the animal.

The invention provides a method of obtaining noninvasive photoplethysmographic measurements from an animal comprising the steps of at least one of [1] using multiple FFTs of different time histories to improve heart and breathing signal strength; [2] using multiple FFTs to get more delta peaks for use in the harmonic heart rate identification method; [3] Basing the heart rate on frequency of side-by-side deltas and/or permutation of all peak deltas; [4] Using of shorter FFTs to reduce the amount of time-history in the frequency spectrum; [5] Zero-padding shorter FFTs to make them the same length as the largest so that all can be compared; [6] Summing or multiply corresponding peaks of FFT to improve signal and skew peaks to more recent time-domain data; providing actigraphy data for the animal.

These and other advantages of the present invention will be clarified in the description of the preferred embodiments taken together with the attached figures.

### BRIEF DESCRIPTION OF THE DRAWINGS

FIG. 1 is a schematic view of a neck mounted non-invasive photoplythosmographic sensor for an awake animal according to the present invention;

FIG. 2 is a representative graph of three Fast Fourier Transforms (FFTs) of the signals from the photoplythosmographic sensor in accordance with the present invention;

FIG. 3 is a representative graph of the AC signals and the DC signals from the photoplythosmographic sensor in accordance with the present invention.

### DESCRIPTION OF THE PREFERRED EMBODIMENTS

In summary, the present invention relates to a noninvasive photoplethysmographic sensor platform for mobile awake animals, such as rats and mice 14 that are utilized in a laboratory environment. Photoplethysmographic measurements on laboratory animals have most often been accomplished on restrained and/or anesthetized animals. This limits the research than can be conducted. Further, in the pulse oximetry field there has been a lack of adequate photoplethysmographic sensors for small mice (and even small rats), until the advent of the Mouse Ox™ brand pulse oximeters by Starr Life Sciences in 2005. Prior to this development, commercially available pulse oximeters could provide heart rate data up to about 350 or 450 beats per minute (and even this range required special software modifications for some sensors), which were basically suitable for rats but not small mice given that the small mouse will have heart rates in the range of 400 to 800 beats per minute. The Mouse Ox™ brand of pulse oximeters for small rodents has an effective range up to about 900 beats per minute as of 2008 models, and later models expands beyond 100 beats per minute with accurate results, which has opened up a wider selection of subjects for this type of research. See also U.S. Patent Publication Numbers 2008-0072906, 2008-0076991, 2008-0132770, 2008-0167564, 2008-0194932, 2008-0262326, 2009-0149727, 2009-0275809 and 2009-0275810 of the assignee, Starr Life Sciences, further describing the operation of a small mammal photoplethysmographic sensors, which publications are incorporated herein by reference in their entirety.

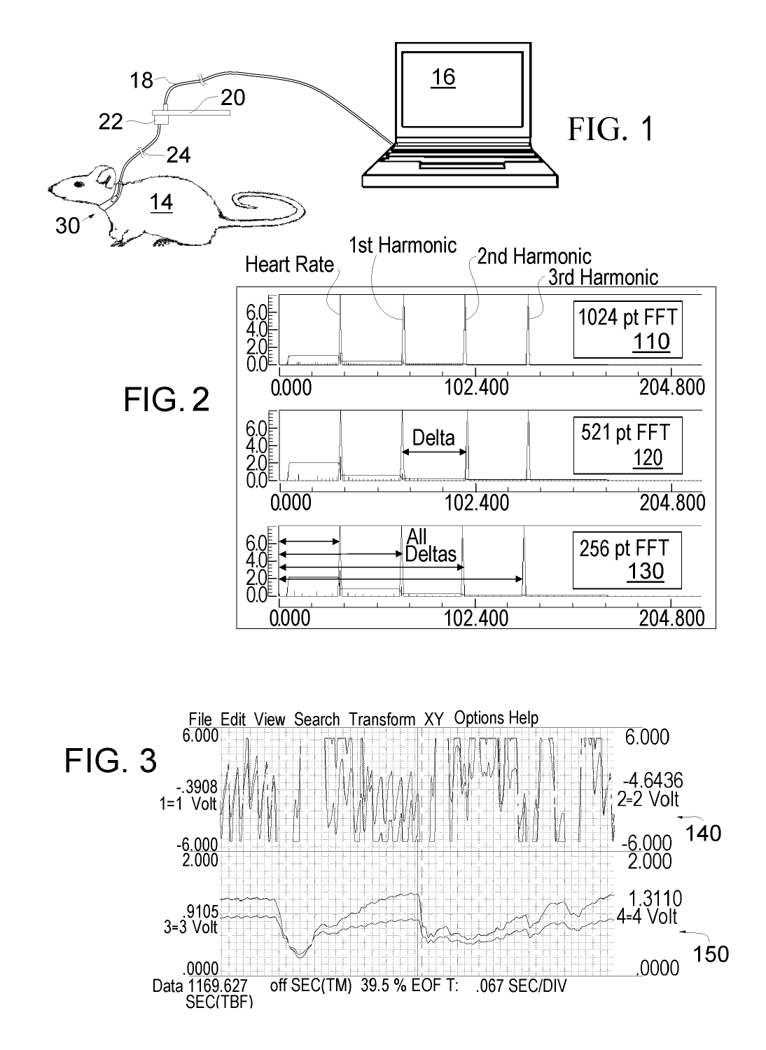

FIG. 1 is a schematic representation of a noninvasive photoplethysmographic sensor for mobile or awake animals such as small rodents 14, namely rats and mice, in accordance with one embodiment of the present invention. The system is particularly well suited for use in a laboratory environment in which a subject animal, such as a mouse 14, is often maintained within a confinement unit (e.g. a cage, cell, housing, etc). The confinement unit is a generic description encompassing anything holding the subject animals 14. The containment unit could be an integral element of the research, such as a maze or other structured test environment. The containment unit will often be a housing area for the animal 14. The details of the containment unit will be well known to those of ordinary skill in animal research fields.

The subject animal 14 may be any subject animal for which photoplethysmographic measurements are desired. A large amount of laboratory research is conducted on rats and mice, however photoplethysmographic measurements has been of limited availability to the researchers when using such subjects. Consequently, the present invention has particular application to research associated with rats and mice. More accurately the present invention provides particular advantages and expands potential research possibilities when utilized with subjects of the order rodentia, and even more precisely, when utilized with the sub-order muroidia. A particularly advantageous aspect of the present invention is that the sensor 30 allows for photoplethysmographic measurements from an awake or even mobile animal 14. The sensor 30 refers in this application to the mounting clip or collar and the associated emitters and receivers for pulse oximetry. The mobile animals 14 may still be retrained by the confinement unit, but the animals 14 may still have a certain range of motion therein. There is nothing that prevents the system of the present invention from being effectively utilized for restrained and/or anesthetized animals 14.

The system will include a processor or controller 16 coupled thereto. The controller 16 is shown schematically in FIG. 1 and can be formed as a laptop or desktop computer. The controller 16 may be the combination of stand alone hardware and software that is coupled with computer for the user interface, display memory and some computation. In this application the controller 16 includes a the user interface, the user display, memory or the like as provided in the commercially available Mouse OX™ product from Starr Life Sciences and is not discussed herein in further detail.

A conventional controller cable 18 extends from the controller 16 for transmitting control and power signals from the controller and data back to the controller 16. The controller cable is coupled to a rotation coupling 20, also called a swivel link. A collar cable 24 is attached to and extends from the rotation coupling 20 through attachment plug 22. The rotation coupling 20 allows relative rotation between the controller cable 18 and the collar cable 24. The rotation coupling 20 provides a convenient location for mounting to the confinement unit. The use of the swivel link or rotation coupling 20 allows the awake animal, e.g. mouse 14, to be effectively freely roaming within the area of the unit 12, wherein twisting of the cables is avoided. The swivel link or rotation coupling 20 also serves to effectively divide the system into an animal specific portion or base and the controller 16, whereby the controller 16 can be easily used with a large number of animal specific portions in a serial fashion. Further, it allows for easy replacement of the portion or base which is anticipated to have a shorter life span than the controller 16.

The present invention does anticipate that the controller 16 may be simultaneously (e.g. a parallel attachment) connected to a number of animal specific portions or sensors 30 through separate cables 18 to allow for obtaining numerous study results at the same time, but this configuration does not eliminate the advantages of the coupling 20.

The sensor 30 includes conventional photoplethysmographic emitters and receivers mounted on a clip member or a body encircling collar configured to encircle a subject animal body portion. Preferably the sensor 30 is configured to be secured around the neck of the subject animal 14. The neck of small mammals such as rats and mice 14 allows for a number of advantages for photoplethysmographic pulse oximetry measurements. The necks of animals of the sub-order muroidia tend to allow for both transmittance and reflective pulse oximetry measurements. Transmittance pulse oximetry is where the received light is light that has been transmitted through the perfuse tissue, whereas in reflective pulse oximetry the representative signal is obtained from light reflected back from the perfuse tissue. Each technique has its unique advantages. Transmittance techniques often result in a larger signal of interest, which is very helpful in small animals that have very small quantities of blood being measured to begin with. Reflective techniques can be used in environments that do not allow for transmittance procedures (e.g. the forehead of a human).

Further, the neck region of the animal offers an area with a relatively large blood flow for the animal, which will improve the accuracy of the measurements. In addition to increased blood flow, the blood flow is present under substantially all conditions. In other areas of the animal, such as the legs, paws and tail, the animal will often cut off blood flow under a variety of conditions. For example if the animal is cold or sufficiently agitated the blood flow to the tail can be shunted. The neck, in contrast represents an area of the animal that will always maintain a constant blood flow for measurements.

The neck also provides a bite proof location for the sensor 30 mounting. In attempting to remove the sensor 30 the biting of most animals, particularly animals of the sub-order muroidia, will be stronger than the clawing, and the neck location prevents the biting attacks as the animal cannot reach the neck collar or neck clip. A secured collar 34 cannot be removed by the animal's paws or clawing.

An alternative location within the scope of the present invention is around the torso, abdomen or chest, of the animal subject. These locations offer some particular advantages and disadvantages. These locations may not provide the same “bite proof” advantages of the neck mounting discussed above, but offer unique pulse oximetry data for small rodents. The abdomen and the chest mounting will not experience blood shunting that can prevent accurate results. Further these locations present particularly advantageous mounting locations for additional sensors, such as accelerometers, EKG leads, temperature sensors and the like.

An alternative location within the scope of the present invention is placing the collar or clip around or on the head of the animal subject with measurements through the head of the animal. The head mounting provides the advantage of being bite proof. It also allows measurements by directing the light through the ears across the head of the animal, which is not a possibility in humans or other large mammals.

One key aspect of the present invention relates to signal processing of a noninvasive photoplethysmographic sensor signal that utilizes multiple FFT's (Fast Fourier Transforms) in the processing of the phtotoplethysmograophic signal, where each FFT has a different signal history, such as by having a different number of points, or by “zero padding” shorter FFTs so they have the same length, as described below.

It had been previously proposed to obtain heart rate from a phtotoplethysmograophic signal from non-moving anesthetized animals by conducting a single FFT on the signal. In the case of heart rate, 1024 points for this FFT as the FFT requires that a “power of 2” number of points be used in the FFT (See 110 FFT of FIG. 3). The single FFT performed on the time-domain signal transfers it into the frequency-domain. Because the original signals are obtained from anesthetized (non-moving) animals, the frequency-domain signal exhibits little noise at frequencies at or near the heart rate and its harmonics. Identification of the heart rate is then done by moving along the FFT from high to low frequency looking for spikes in the FFT signal that pass a given threshold. When a spike or peak is found, the threshold is increased and the search is continued in the direction of low frequency. The assumption is that the because of low noise at the higher end of the spectrum, the only spikes present in the FFT at frequencies above the heart rate are harmonics of the heart rate. Thus, the frequency locations of these peaks, and more importantly, the difference in frequency between them, are collected. If any two peaks are truly harmonics of the heart rate, the difference in frequency between them (a “delta”) IS the heart rate, since harmonics are simply integer multiples of the fundamental frequency, which is the heart rate. Validation of this method is done by looking for matching deltas.

The approach described above works very well for finding heart rates on animals that are immobile. However, new sensor designs allow measurements to be made on spontaneous breathing, freely roaming rodents. Freely roaming rodents move almost continuously, and this motion translates to large frequency content on the FFT, which can overwhelm the peak detection method described above.

In the method according to the present invention, both a 512 point (120) and a 256 point (130) FFT are used for heart rate identification, in addition to the 1024 point FFT. The benefit is that a 512 point FFT 120 contains half the history of the 1024 FFT 110, and a 256 FFT 130 contains one quarter of that in the 1024 FFT 110, allowing a motion spike to clear 2 and 4 times more quickly, respectively. Note that smaller or even larger FFT sizes could be used as well.

After all of the deltas are calculated from each FFT, they are sorted numerically. The number of matching deltas is then recorded for each different delta value (note that delta values are in bin numbers, so they are all integers). In addition, the number of matching deltas plus all of those that are one behind or one in front of the given delta are recorded. The number of all matching deltas +/−1 is then summed with the number of all matching deltas alone. The largest value of this sum is then considered to be the location of the heart rate.

Another application for multiple FFTs is to zero-pad them to the same length as described above. It is then possible to either sum the magnitudes of the FFTs at each bin location, or to multiply them together at each non-zero bin location. The idea is to improve the signal to noise ratio by increasing the amplitude of significant signal locations without increasing the noise floor, and to increase representation of more recent time-domain information in the frequency spectrum.

It is understood that motion of the animal can disrupt the reliability of photoplethysmographic pulse oximetry measurements. The commercially available Mouse Ox™ system, which has been used for thigh and tail sensor mounted applications for restrained or anesthetized animals, has developed motion artifact rejection techniques to avoid inaccurate readings associated with tail or paw motion or the like. This motion artifact data rejection technique becomes even more important with mobile animals. Further, the existing techniques are sufficient to allow for reasonable number of readings in a mobile animal.

Photoplethysmographic signals that are received from an animal while it is moving exhibit large swings in amplitude that are difficult from which to make measurements. Typically, such signals often hit the amplifier limits, but at least are VERY large compared to correctly sized and shaped signals from which measurements can be made. Additionally, these large swings can cause the signal strength controller to react by reducing amplification of the signals so that they do not rail the amplifiers. Since motion of the animal is their cause, it is generally undesirable to adjust the controller during these motions. The method proposed here is designed to set a flag when motion is detected so that the controller does not adjust and measurements are not made in response to motion. A second important use of this motion detection is to track how much the animal is moving, herein after referenced as “actigraphy” measurements.

The following method is one actigraphy determining methodology, other motion artifact determining algorithms may be utilized to provide actigraphy measurements. One method used to generate an error flag that can identify large swings in photoplethysmographic signal amplitude resulting from animal motion starts by calculating the difference between the maximum and minimum values of a group of digitized photoplethysmographic data. It can be summarized as follows: Use of the red and infrared DC photoplethysmographic signals for identification of abnormally large spikes that occur with motion; Measuring the max and min values in a span of data points to characterize their noise level; Making a comparison of the these max and min values from one set of data to the next, and checking if their relative difference surpasses a set threshold; and Setting a flag if the threshold is passed so that light amplification and parameters calculation may be ceased while the flag is active. In the system used here, the number of points in the proposed sample group is 217, which is the number of samples (sampled at 300 Hz) that are passed from the AID card buffer to the processor. With each group of 217 points, a delta is calculated between the minimum and the maximum, which we will denote Δ.

This calculation of Δ is made on both red and infrared DC signals 160. DC signals 160 are the relatively unamplified raw photoplethysmographic signals that are received from both the red and infrared LED emitters. The use of DC signals 160 in this algorithm is important because clean DC values 160 typically are very flat and only drift slowly. Additionally, the amplitude variation resulting from changes in arterial blood volume between the sensor pads due to the cardiac stroke is so small that it is often not visually discernable on the DC signals 160. They may be as little a 1/100^{th} to 1/1000^{th} of the size of the average DC signal amplitude. Conversely, motion of the animal causes spikes that are anywhere from 2 to 10 times or greater than the typical DC signal 160. On the AC signals 150 (high-pass filtered, highly amplified versions of the DC signals), these motion spikes simply quickly become flat as the amplifiers are saturated, rendering identification of motion noise not discernable from improper photopleth signal amplification. Thus, motion is very obvious when it occurs, but only on the DC signals 160. The method described here is conducted on the DC signals 160 only.

Regarding other actions taken when a motion is detected (new Δ is at least 3 times the old value), the controller stops adjusting amplification of the photopleth signal. Also, no parameter calculations are made during that time that the motion flag is active. Maintaining a measurement of when the motion flag is active and how much it is active will provide a useful actigraphy measurement from the pulse oximeter of the present invention.

Motion of the animal may not eliminate the ability to obtain meaningful data, consequently it is possible for the actigraphy measurements (the motion detecting algorithms) to have multiple settings of detected animal movement. A first lower detected animal movement level will merely indicate the animal is moving and set an actigraphy flag or marker. A higher level of motion could be identified that both indicates motion of the animal and stops parameter calculation due to possible error in calculations as described above.

The actigraphy measurement need not be limited to the DC signal review described above, but any motion artifact detection algorithm could be utilized. For example, a change in the pulse distension baseline could be used as a movement measurement as this is typically a result of relative motion of the sensor to the animal. An alternative motion detecting algorithm is via calculating the area under the AC signal FFT plot and using this as an indicator of animal movement. An alternative motion detecting algorithm is reviewing the railheads in the AC signal with a present number of railhead indicating movement. Further, reviewing the overall oscillation of the DC signal could be utilized as a motion detection indicator, wherein a change in DC signal average is likely due to animal movement and slippage of the sensor 30. All of these can be used to identify the animal motion and track actigraphy of the amount of movement of the animal. It is further contemplated that some of these parameters would allow a gradient of motion to be calculated to give a meaningful measurement of the type of animal motion that is being exhibited by the animal rather than merely tracking the presence of some type of movement over time.

With regard to the display of the actigraphy measurements, it is expected the motion detecting flags or indicators in the system will be sent to a buffer before display to the user such that the presence of multiple flags over a series of time could be used as a real actigraphy indication. In other words more than one consecutive “motion flag” may be used to indicate the presence of actual or continuous animal motion (although the presence of one significant flag may prevent the calculation of selected parameters for a minimal time).

Whereas particular embodiments of the invention have been described above for purposes of illustration, it will be evident to those skilled in the art that numerous variations of the details of the present invention may be made without departing from the spirit and scope of the present invention.

What is claimed is:

1. A noninvasive photoplethysmographic sensor for small animals wherein the sensor is configured to utilize multiple FFT's of the photoplethysmographic sensor signals in the processing of the photoplethysmographic signals, where each FFT has a different amount of phtotoplethysmographic signal time history.

2. The noninvasive photoplethysmographic sensor for small animals according to claim 1 wherein each FFT has a different number of points.

3. The noninvasive photoplethysmographic sensor for small animals according to claim 2 wherein three distinct FFTs of the photoplethysmographic sensor signals are utilized in the processing of the photoplethysmographic signals.

4. The noninvasive photoplethysmographic sensor for small animals according to claim 3 wherein three distinct FFTs include a 1024 point FFT, a 512 point FFT and a 256 point FFT.

5. The noninvasive photoplethysmographic sensor for small animals according to claim 1 wherein the deltas between identified peaks of each FFT is used to calculate heart rate.

6. The noninvasive photoplethysmographic sensor for small animals according to claim 1 wherein the peaks of different FFTs are mathematically combined to improve signal to noise ratio.

7. The noninvasive photoplethysmographic sensor for small animals according to claim 6 wherein the peaks of different FFTs are summed.

8. The noninvasive photoplethysmographic sensor for small animals according to claim 6 wherein the peaks of different FFTs are multiplied.

9. The noninvasive photoplethysmographic sensor for small animals according to claim 6 wherein shorter FFTs are zero padded to form all the FFTs of the same length for comparison.

10. The noninvasive photoplethysmographic sensor for small animals according to claim 1 further including providing actigraphy measurements from the sensor.

11. The noninvasive photoplethysmographic sensor for small animals according to claim 10 further including the identification of large spikes in the DC signal as animal motion indicators for actigraphy measurement.

12. The noninvasive photoplethysmographic sensor for small animals according to claim 10 wherein other physiologic parameter calculations are postponed if the actigraphy measurement exceeds a certain threshold.

13. A noninvasive photoplethysmographic sensor for small animals wherein the sensor is configured to provide actigraphy measurements of the animal from the sensor.

14. The noninvasive photoplethysmographic sensor for small animals according to claim 13 further including the identification of large spikes in the DC signal as animal motion indicators for actigraphy measurement.

15. The noninvasive photoplethysmographic sensor for small animals according to claim 13 wherein other physiologic parameter calculations are postponed if the actigraphy measurement exceeds a certain threshold.

16. The noninvasive photoplethysmographic sensor for small animals according to claim 13 wherein the sensor is configured to utilize multiple FFT's of the photoplethysmographic sensor signals in the processing of the photoplethysmographic signals, where each FFT has a different number of points.

17. The noninvasive photoplethysmographic sensor for small animals according to claim 13 wherein the sensor is configured to utilize multiple FFT's of the photoplethysmographic sensor signals in the processing of the photoplethysmographic signals.

18. The noninvasive photoplethysmographic sensor for small animals according to claim 17 wherein the peaks of different FFTs are mathematically combined.

19. The noninvasive photoplethysmographic sensor for small animals according to claim 17 wherein the deltas between identified peaks of distinct FFTs is used to calculate physiologic parameters of the animal.

20. A noninvasive neck mounted photoplethysmographic sensor for a mouse wherein the sensor is configured to utilize multiple FFT's of the photoplethysmographic sensor signals in the processing of the photoplethysmographic signals, and wherein the wherein the sensor is configured to provide actigraphy measurements of the mouse from the sensor.
